(19) United States
(12) Patent Application Publication
(10) Pub. No.: US 2008/0311054 A1
(43) Pub. Date: Dec. 18, 2008

(54) Compositions and Methods to Counteract Oral Malodour

(75) Inventors: Andreas Natsch, Uetikon (CH); Eduardo Moraes, Zurich (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 12/064,506

(22) PCT Filed: Aug. 24, 2006
(86) PCT No.: PCT/CH2006/000460

§ 371 Date: Feb. 22, 2008

(30) Foreign Application Priority Data
Aug. 30, 2005 (GB) 0517577.3

(51) Int. Cl. A61K 8/37; A61K 31/22; A61K 36/82; A61K 36/534; A61Q 11/00

(52) U.S. Cl. 424/48; 514/549; 424/729; 424/747; 424/49

(57) ABSTRACT

The invention relates to hedonistically pleasing oral malodour counteracting compositions including flavour compositions and oral care products, methods to form such compositions and methods to counteract oral malodour. The compositions comprises 2 or more of oral malodour counteracting actives of formula I in a total concentration of at least 10% (w/w) based on the total concentration of flavour ingredients, and an individual concentration of 1% or more per oral malodour counteractant, based on total flavour ingredients. The maximum concentration of any individual oral malodour counteracting active based on the total of oral malodour counteracting actives is 70%. The identified oral malodour counteracting actives are various flavour compounds and natural ingredients.

The present invention is directed to methods of counteracting oral malodour, to oral malodour counteracting (OMC) compositions comprising OMC actives, and to oral care products formed by admixing said OMC actives or compositions to an oral care product formulation.

The key volatiles involved in oral malodour include various sulphur molecules, especially hydrogen sulphide (H₂S), methanethiol (MeSH) and dimethylsulfide (Me-S-Me). Among these, MeSH has the lowest odour threshold, and is therefore of highest relevance.

Oral care products, for example toothpaste, mouth rinse, and chewing gum, classically contain intense flavours to mask oral malodour, or rather its perception, by using a dominating flavour or odour, while the malodour remains present but is less detectable in combination. For example, JP 2004018431 describes various flavour compositions comprising mint oils or compounds known to be comprised in mint plants, which are known actives against halitosis (for example menthol), in combination with masking flavour compounds.

Oral maldodour is formed by gram-negative bacteria in the mouth. Another classical approach to reduce oral malodour is therefore to combat these bacteria, for example by classical antibacterial agents such as Triclosan, cetyl-pyridinium chloride, and chlorhexidine.

In some cases the antibacterial effect of natural ingredients or flavour compounds is used. These include, for example, thymol, wintergreen oil, methyl salicylate, eucalyptol and mint oils and compounds occurring in mint plants, in particular menthol. Further natural ingredients that are known to have a malodour counteracting effect include parsley, which has been used since ancient times against oral malodour. A combination of ionones with zinc salts has also been used to counteract oral malodour (alpha-ionone, beta-ionone, gamma-ionone, dihydroionone, alpha-methylionone, irone). Furthermore, certain higher alcohols, in particular nonanol, are known to kill microorganisms such as yeasts and to be useful in oral care compositions when combined with C1-C4 lower alcohols (WO 99/51093). Notably, octanol was found to have no effect.

However, the complete inhibition or eradication of these bacteria may be impossible and often is unwanted in order not to disturb the naturally-occurring oral bacteria that, when disturbed, may potentially be replaced by more harmful microorganisms.

An alternative is to reduce oral malodour by means that leave the oral bacteria largely intact, in particular by chemically capturing the malodorous volatiles by reactive chemicals. For example polyphenolic compounds such as those contained in green tea extract have been shown to capture volatile sulphur compounds. The same mode of action is also attributed to zinc salts regularly used in oral care products. A further chemical approach is to degrade the malodorous sulphur volatiles by applying oxidizing agents. However, the drawback of these chemical approaches is that for each molecule of the odoriferous sulphur compound, a stoichiometric amount of the binding or degrading molecules is needed, and therefore relatively high concentrations of the reactive chemicals are necessary to successfully counteract oral malodour.

Another approach is by enzymatic inhibition of the relevant bacterial enzyme(s) so that the malodorous sulphur volatiles are not formed in the first place. For example, certain plant extracts (tomato, Uncaria gambier, Quillaja saponaria, Hamamelis virginiana, Eriobotrya japonica, Equisetum arvense, Crataegus oxyacantha, Diospyros kaki, Curcuma domestica, Ginkgo biloba, green tea, black tea, and/or oolong tea) are known to inhibit the methioninase enzyme which generates MeSH. For example, a mouth wash is known containing tomato ext. 0.001, cinnamic aldehyde 0.0001, cetylpyridinium chloride 0.0001, chlorhexidine gluconate 0.0001, polyoxyethylene hydrogenated castor oil 2, glycerol 8, ethanol 5, sodium saccharin 0.04, and water q.s. to 100%.

All the above described approaches are only partially successful in their oral malodour counteracting effect, in particular they suffer the following drawbacks. The masking by adding flavour approach will not completely mask the malodour and is of short duration. Antibacterial agents reduce the oral bacterial population, but completely disinfecting the oral cavity is not possible or necessarily wanted. The chemical binding or degradation needs a high amount of active and therefore is inefficient and not practical.

A further drawback with the use of known ingredients, for example thymol, eugenol, cinnamic aldehyde and menthol, is that they have a dominating flavour when used at the relatively high concentration that is needed for effectiveness, which then leads to a product hedonically unpleasant and not readily accepted by the consumer. Further, many actives, in particular antibacterial actives, have a bitter or astringent taste, for example Triclosan or zinc salts.

With the currently known oral malodour counteracting ingredients, it is difficult for flavourists to provide oral care products with both sufficient activity and an acceptable flavour. This is particularly difficult for ingredients that need to be integrated into a composition in a high concentration to have a sufficient oral malodour counteracting effect. Many antibacterial compounds or other actives have unpleasant tastes. Flavour compounds or ingredients containing a predominant flavour compound will, when used in a high concentration, result in a taste perceived as “chemical” or overpowering by consumers. When a lower concentration is used to avoid the unpleasant taste, this concentration may not be sufficient to effectively counteract oral malodour. An overpowering unpleasant taste is in particular the case for thymol, eugenol, cinnamic aldehyde and menthol. Menthol will furthermore elicit a burning sensation at higher concentration while at lower concentration it is perceived as cooling (both effects are mediated via the trigeminal nerve rather than via flavour receptors). While accepted by some consumer groups, others, and in particular children, are more sensitive, especially to menthol. Therefore, in particular for certain products including children's toothpaste, an alternative is needed.

Therefore there remains a need for hedonistically acceptable compositions providing a sufficiently high malodour counteracting effect.

An additional problem when identifying ingredients for such compositions is an adequate testing system. Applicant has found that while certain ingredients appear to sufficiently inhibit enzymes in vitro and in tests using single species of bacteria, often the results are different once the ingredient is tested on whole-tongue bacterial populations in saliva/tongue scraping samples, as these ex vivo samples include various different species of bacteria and better represent the in vivo situation. Therefore, it is important to test the effectiveness of oral malodour counteracting actives on mixed bacterial populations of the tongue instead of (or in addition to) tests on single relevant bacteria species such as F. nucleatum. Furthermore, the mixed bacterial sample from the tongue further contains all the proteins from saliva which may adsorb active ingredients, and it contains the hydrolytic enzymes of the saliva, which may inactivate certain ingredients, for example esters. The experiments and results are described in detail in the examples.

Using the abovementioned test systems, surprisingly, a structural group of food-grade ingredients including flavour compounds have been identified as oral malodour counteracting actives useful in flavour compositions or oral care products according to the invention. These identified oral malodour counteracting ingredients or compounds (“OMC actives”) are found to have a sufficient malodour counteracting activity so that flavour compositions and oral care products can be formed that do not have an overpowering flavour.

OMC actives useful for flavour compositions and oral care products according to the invention are compounds according to formula I

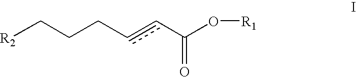

wherein
R₁ is a residue selected from the group consisting of CH₃ or CH₂CH₃;
R₂ is a residue selected from the group consisting of H, CH₃, CH₂CH₃, CH₂CH₂CH₃; and the double bond with dashed line as shown is a double bond or a triple bond.

OMC actives as defined herein above allow the flavourist to provide an effective OMC composition with a flavour readily accepted by the consumer when used in combination of at least 2 OMC actives in a OMC composition. A hedonistically even more pleasant OMC composition can be achieved when using 3 or more OMC actives.

The present invention therefore provides compositions that are both highly effective against oral malodour and at the same time hedonistically pleasant for the consumer. Further the invention provides methods to form said OMC compositions and methods to counteract oral malodour by employing said OMC compositions.

OMC compositions according to the invention may be used to reduce the concentration of known oral malodour counteractants while keeping the OMC effect of the composition, or to enhance the effect of a composition with known oral malodour counteractants at a given concentration.

In a first aspect, the invention is directed to a composition comprising

(a) 2 or more oral malodour counteracting actives,
wherein of said 2 oral malodour counteracting actives each has a concentration of 1% (w/w) or more based on total flavour ingredients, and
wherein the total concentration of the 2 or more oral malodour counteracting actives is from 10% (w/w) or more based on the total concentration of flavour ingredients, and
wherein each individual OMC active has a maximum concentration of up to 70% based on the total concentration of OMC actives, and
(b) optional ingredients selected from additives, excipients, solvents, and flavour ingredients;
wherein the 2 oral malodour counteracting actives are compounds according to formula I

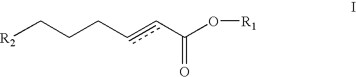

wherein
R₁ is a residue selected from the group consisting of CH₃ or CH₂CH₃;
R₂ is a residue selected from the group consisting of H, CH₃, CH₂CH₃, CH₂ CH₂CH₃; and the double bond with dashed line as shown is a double bond or a triple bond.

In a particular embodiment, OMC actives are selected from the group consisting of Oct-2-ynoic acid methyl ester, Non-2-ynoic acid methyl ester, Oct-2-enoic acid ethyl ester, Oct-2-enoic acid methyl ester, Non-2-enoic acid methyl ester, Hex-2-enoic acid ethyl ester, Hex-2-enoic acid methyl ester, Non-2-ynoic acid ethyl ester, Non-2-enoic acid ethyl ester, Hept-2-enoic acid ethyl ester, and Hept-2-enoic acid methyl ester.

In another embodiment, the OMC composition comprises at least 2 of the 2 or more OMC actives selected from the particular group of OMC actives as defined in the previous paragraph. The OMC composition may also comprise 2 OMC actives selected from this group, and may or may not comprise additional OMC actives.

In another embodiment, a OMC composition as defined herein further comprises as an additional component one or more actives selected from the group consisting of ionone, alpha ionone, beta ionone, zinc salts, polyphenolic compounds, and antibacterial agents.

Antibacterial agents may be selected from the group consisting of triclosan, cetylpyridinium chloride, polyhexidine bisguanide, chlorhexidine, and antibacterial flavour materials. Antibacterial flavour materials include in particular thymol, carvacrol, eugenol, isoeugenol, cinnamic aldehyde, menthol. Flavour materials may be provided in form of an essential oil containing these ingredients. Preferred essential oils include oil from thyme, origanum, clove, cinnamon leave, cinnamon bark, parsley seed, parsley leaf, mint, spearmint, and peppermint.

Useful polyphenolic compounds are, for example, those that comprise a gallate moiety, in particular epigallocatechin gallate. These may be in form of certain natural ingredients, in particular in green tea and its extract, for example green tea extract enriched in epigallocatechin gallate. In particular, an OMC flavour in particulate form may be formed by spray-drying an OMC flavour composition and mixing it with green tea particles to form a dry blend of green tea and OMC flavour composition. The resulting particulate material can be easily admixed to an OMC product formulation.

In another embodiment, a OMC composition as defined herein further comprises as an additional component one or more actives selected from the group consisting of 5-Isopropyl-2-methyl-phenol, Octan-1-ol, 3,7-Dimethyl-oct-6-en-1-ol, 3,7-Dimethyl-octan-1-ol, 1-Isopropyl-4-methyl-cyclohex-3-enol, 3,7-Dimethyl-octa-2,6-dien-1-ol, 2-(4-Methyl-cyclohex-3-enyl) propan-2-ol, 3,7-Dimethyl-octa-1,6-dien-3-ol, Nona-2,4-dienal, Non-2-enal, 2,6,6-Trimethyl-cyclohex-1-enecarbaldehyde, 3-(4-Isopropyl-phenyl)-2-methyl-propionaldehyde, 4-Isopropenyl-cyclohex-1-enecarbaldehyde, 5-Methyl-2-phenyl-hex-2-enal, 4-Methoxy-benzaldehyde, 2,6-Dimethyl-hept-5-enal, Dec-2-enal, Phenyl-acetaldehyde, 2-Phenyl-propionaldehyde, 3,7,11-Trimethyl-dodeca-1,3,6,10-tetraene, 3,7-Dimethyl-octa-1,3,6-triene, 1-Isopropyl-4-methyl-cyclohexa-1,3-diene, 1-Methyl-4-(5-methyl-1-methylene-hex-4-enyl)-cyclohexene, 1-isopropyl-4-methylbenzene, Dec-3-en-2-one, 3-Methyl-2-pentyl-cyclopent-2-enone, 6-Methyl-hepta-3,5-dien-2-one, Acetic acid octyl ester, Acetic acid oct-2-enyl ester, 2-Methyl-but-2-enoic acid hex-3-enyl ester, Acetic acid nonyl ester, Acetic acid heptyl ester, Butyric acid 3-phenyl-allyl ester, Acetic acid 1,7,7-trimethyl-bicyclo[2.2.1]hept-2-yl ester, Acetic acid 4-allyl-2-methoxy-phenyl ester, Acetic acid 1-methyl-1-(4-methyl-cyclohex-3-enyl)-ethyl ester, Acetic acid 2-isopropenyl-5-methyl-cyclohexyl ester, 5-Octyl-dihydro-furan-2-one, 1,1-Dimethoxy-3,7-dimethyl-octa-2,6-diene, 1-Allyl-4-methoxy-benzene, 6-Hexyl-tetrahydro-pyran-2-one, 3-Butyl-3H-isobenzofuran-1-one, 2-Pentyl-furan, (2E, 5E/Z)-5,6,7-trimethylocta-2,5-dien-4-one, 4-methyl-dec-3-en-5-ol, 1-cyclopropylmethyl-4-methoxy-benzene, origanum essential oil, galbanum essential oil, litsea cubeba essential oil, tagete essential oil, jasmin absolute, lavande essential oil, lavandin essential oil, rosemary essential oil, and vetiver essential oil.

The combination of enzyme-inhibiting oral malodour counteracting substances with certain antibacterial flavors has been found to be particularly beneficial and results in compositions both highly effective and pleasantly tasting. Accordingly, in another embodiment, a OMC composition as defined herein further comprises as an additional component of up to 50%, or up to 90% (w/w) of total flavor ingredients, one or more flavors with antibacterial properties selected from the group consisting of menthol, thymol, eugenol, 5-Isopropyl-2-methyl-phenol, Octan-1-ol, 3,7-Dimethyl-oct-6-en-1-ol, 3,7-Dimethyl-octan-1-ol, 1-Isopropyl-4-methyl-cyclohex-3-enol, 3,7-Dimethyl-octa-2,6-dien-1-ol, 2-(4-Methyl-cyclohex-3-enyl) propan-2-ol, and 3,7-Dimethyl-octa-1,6-dien-3-ol.

The compounds can be added in the form of pure compounds or in the form of natural ingredients (for example essential oils from plants, such as mint oils, peppermint oil, spearmint oil for menthol or thyme oil for thymol).

In another embodiment, the total concentration of the 2 or more OMC actives is at least 20%, 30%, 40%, 50%, 60%, or at least 70% (w/w) or more based on the total concentration of flavour ingredients in the OMC composition (excluding excipients such as solvents and additives).

In another embodiment, the OMC composition is as defined above, and each individual OMC ingredient has a maximum concentration of up to 60%, 50%, 40%, 30% and 20%, which will exceedingly avoid a flavour perceived as “chemical”, “artificial”, unpleasant, overpowering or unbalanced.

For a composition particularly well-balanced in its flavour, 3 or more OMC actives are used.

Compositions according to the invention comprise OMC flavour compositions and OMC oral care products. OMC flavour compositions may be added to oral care formulations to form an OMC oral care product. Alternatively, OMC compounds may be directly added to an oral care product formulation to form an OMC oral care product.

In another aspect the invention is directed to a method of forming an OMC composition by admixing 2 or more OMC actives (optionally in form of an OMC flavour composition as described herein above) to an oral care formulation to form an OMC oral care product in a concentration of about 0.05 to about 1% (w/w) of OMC actives as described herein above based on total weight of the OMC oral care product.

In yet another aspect the invention is directed to a method of counteracting oral malodour by orally applying an OMC oral care product as defined herein.

To provide a sufficient OMC effect in oral care products, the OMC actives or OMC flavour compositions are used in a concentration so that the total concentration of OMC actives in the oral care product provides sufficient OMC activity, for example in the concentrations given below. Oral care products comprise OMC actives as defined herein above in a total concentration w/w from about 0.05% to about 1%, from about 0.1% to about 0.75%, or from about 0.15% to about 0.4% (w/w based on total weight of the oral care product). The latter concentration in particular provides at the same time an excellent activity and a flavour that is perceived as pleasant by the consumer. The indicated higher concentrations improve the activity while providing a flavour still readily accepted by most consumers. The given lower concentrations provide an excellent flavour with an activity still sufficiently effective.

Oral care products may be formed by addition of the above-defined OMC actives or OMC flavour compositions to known oral care product formulations. Oral care products include, for example, toothpaste, mouthrinse, mouthwash, and portable “on the go” oral malodour control products including chewing gum, candies, pastilles, edible films, and oral sprays. Formulations for the above-mentioned oral care products are well-known in the art. Oral care products contain excipients including, for example, surfactants, emulsifiers, solvents, colorants, preservatives, antioxidants, antimicrobial agents, enzymes, vegetal or mineral oils, fats, proteins, solubilisers, sugar derivatives, vitamins, polyols including sorbitol, organic acids, artificial sweeteners, polymers, thickeners, chewing gum gum bases, oral care actives including fluorine compounds, and zinc salts (for example zinc gluconate, zinc acetate, zinc citrate). Some oral care products contain alcohols, in particular lower alcohols (C1-C4). The compounds of the present invention are not dependent on the presence of a lower alcohol for their activity and will be active in water-based composition without C1-C4 alcohols. Advantageously, compositions without alcohols, in particular without lower alcohols (C1-C4) can be formed. This is desirable for example to avoid the drying-out effect these alcohols may have on epithelia.

For particular oral care products, certain concentration ranges may be chosen to provide at the same time a good activity and a flavour perceived as pleasant.

For example, for toothpaste, a concentration from 0.1% to 0.625%, or from 0.25% to 0.4% (w/w total volume), of OMC actives as defined herein above is useful.

For example, for mouthwash, a concentration from 0.05% to 0.25%, or from 0.075 to 0.125% (w/w total volume), of OMC actives as defined herein above is useful.

For example, for chewing gum, a concentration from 0.1% to 0.75%, or from 0.2% to 0.625% (w/w total volume), of OMC actives as defined herein above is useful.

OMC compositions may comprise additional ingredients and excipients well known in the art, in particular additional flavour ingredients to provide a desired flavour accord. Examples of known flavour ingredients may be found in one of the FEMA (Flavour and Extracts Manufacturers Association of the United States) publications or a compilation thereof which is available from and published by FEMA and contains all FEMA GRAS (generally regarded as safe) publications, 1965-present, in particular publications GRAS 1-21 (the most recent one being GRAS 21 published 2003), or in Allured's Flavor and Fragrance Materials 2004, published by Allured Publishing Inc. Examples of know excipients for oral care products may be found in Gaffar, Abdul, Advanced Technology, Corporate Technology, Department of Oral Care, Colgate-Palmolive Company, Piscataway, N.J., USA. Editor(s): Barel, Andre O.; Paye, Marc; Maibach, Howard I., Handbook of Cosmetic Science and Technology (2001), 619-643. Publisher: Marcel Dekker, Inc., New York, N.Y., and in Cosmetics: Science and technology, 2nd edition, p. 423-563. Edited by M. S. Balsam and E. Sagarin, Wiley Interscience, 1972.

### EXAMPLES

### Example 1

### Identification of Potential OMC Actives by In-Vitro Screening for Enzyme Inhibition

The enzyme methionine-γ-lyase cleaves methionine into methanethiol (MeSH), ammonium and α-keto butyrate. Genomic DNA is extracted from a bacterium expressing the above enzyme, for example Fusobacterium nucleatum, strain DSMZ 20482 (publicly available from Deutsche Sammlung Microorganismen und Zellkulturen, Braunschweig). The gene coding for the methionine- [symbol] -lyase is amplified using appropriate primers which will differ depending on the bacterium. For Fusobacterium nucleatum, the following primers are used: CATGCCATGGAAATGAAAAAATCTGGT and CGGAATTCCCAATTTTTTCTAGTCCTTGTTC, employing standard PCR conditions with reagents obtained from SIGMA (Buchs, Switzerland). The amplified region is purified and digested with the restriction enzymes NcoI and EcoRI. The open reading frame is then ligated to a sequence coding for a 6× Histidine-Tag and cloned into the expression vector pET-3a (Studier and Moffatt, 1986). The resulting plasmids are transformed into the E. coli host strain BL21 (DE3). The recombinant strains are grown in a standard growth medium (LB), induced with IPTG (isopropyl-beta-D-thiogalactopyranoside) and after 4 h the cells are lysed by three passages through a french press in a phosphate buffer (50 mM, pH8) containing 10 mM imidazole. The cell lysate is cleared by centrifugation at 10'000 g for 15′ and the supernatant is loaded onto a Ni-NTA affinity column (Qiagen, Hilden, Germany). The column is washed with the same buffer but containing 20 mM imidazole and finally eluted with the same buffer but with a concentration of 250 mM imidazole. The resulting eluate contains the recombinant enzyme in >90% purity, and is used for screening assays as follows:

Ingredients or compounds to be tested are dissolved in DMSO to a final concentration of 4% and serially diluted in the same solvent. Aliquots of the solutions of different inhibitors (2.5 μl) are distributed to individual wells of a microtiter plate. The recombinant enzyme is diluted 20 fold in 50 mM phosphate buffer, pH 7 (Buffer A) and 100 μl are added to each well. The reaction is started by adding the substrate methionine (100 μl, concentration of 2 mM in Buffer A). After 1 h of incubation, the released MeSH is derivatised by adding to each well of the microtiter plate 100 μl of a monobromobimane (obtained from Fluka, Buchs, Switzerland) stock solution (0.5 mM in 1 M NaCO₃, pH 8.8). After 10 min the fluorescence in the wells of the microtiter plates is measured on a Flex-station (Molecular devices, Sunnyvale, Calif., USA) with an excitation wavelength of 385 nm and an emission wavelength of 480 nm. After the fluorescence determination, from all the wells the blank value containing only buffer, DMSO and the enzyme without added substrate is subtracted. The fluorescence of control wells with enzyme, substrate and DMSO only is then compared to the fluorescence in wells containing potential inhibitors to calculate the inhibition in percent. Table 1 herein below list the identified OMC actives that inhibit the enzyme and that are useful in a composition according the invention. Identified OMC actives have an IC 50 (concentration giving 50% enzyme inhibition) of less than 0.01% weight per volume, and are active also in mixed bacteria cultures grown from saliva samples, see example 3.

### Example 2

### Identification of Potential OMC Actives by In-Vitro Screening Method for Inhibitors of Mesh Formation in F. nucleatum Cultures

F. nucleatum DSMZ 20482 is grown on agar plates containing medium 104 (German collection of microorganisms and cell cultures, Braunschweig, Germany) for 48 h under anaerobic conditions at 37° C. Cells are harvested and suspended in a phosphate buffer (50 mM, pH7) containing 2.92 g/L NaCl to a final optical density of 1 measured at 600 nm. Anaerobic conditions are generated by applying a nitrogen stream to the cell suspension and the substrate methionine is added to a final concentration of 1 mM. Test compounds are dissolved in DMSO to a concentration of 2% (w/v) and 10 μl of the solution is added to a 5 ml headspace GC vial. The vials are sealed and oxygen removed by applying a nitrogen stream. To each vial, 1 ml of the methionine containing cell suspension is added and the cultures are incubated for 4 h at 37° C. The cultures are then pasteurized by heating to 80° C. for 15′ and the level of MeSH in the headspace is determined by gas chromatography: Samples are heated to 75° C. and 1 ml of the headspace is injected onto a column suitable for separation of sulphur compounds (SPW1-sulfur, Supelco). The temperature program is set to 1 min initial temperature at 50° C., heating at a rate of 10° C./min to 100° C. and further heating at 20° C./min to 200° C.

The concentration of formed MeSH is compared to control cultures containing DMSO solvent only, and % inhibition of MeSH formation is calculated. Identified OMC actives have an average % inhibition of at least 60% when tested at 0.02% concentration (w/w), and are included in Table 1, which list the identified OMC actives useful in compositions according to the invention that inhibit the enzyme shown by method of example 1 and/or 2, and are active also in mixed bacteria cultures grown from saliva samples, see example 3.

### Example 3

### Identification of Potential OMC Actives by Ex-Vivo Screening Method for Inhibitors of MeSH Formation in Mixed Bacterial Cultures Grown from Incubated Saliva

Saliva samples are collected from panelists who are instructed to simultaneously harvest the bacterial biofilm of their tongue by scratching over their tongue with their teeth. The harvest containing saliva and bacteria from four donors is pooled, diluted with a phosphate buffer (50 mM, pH7) containing 2.92 g/L NaCl in a ratio of 2:1 and supplemented with methionine to a final concentration of 1 mM. Test compounds are dissolved in DMSO to a concentration of 2% (w/v) and 10 μl of the solution is added to a 5 ml headspace GC vial. The vials are sealed and oxygen removed by applying a nitrogen stream. To each vial, 1 ml of the methionine containing saliva sample is added and the cultures are incubated for 4 h at 37° C. The cultures are then pasteurized and the level of MeSH in the headspace is determined as described above.

The concentration of formed MeSH is compared to control cultures containing DMSO solvent only, and % inhibition of MeSH formation is calculated. Table 1 list the identified MOC actives that inhibit MeSH formation. Identified OMC actives have an average % inhibition of at least 50% when tested at 0.02% concentration (w/w).

TABLE 1
OMC actives identified as described in examples 1-3

| IUPAC name | Trivial name or alternative names |
|---|---|
| oct-2-ynoic acid methyl ester | methyl octinoate, Folione™ |
| non-2-ynoic acid methyl ester | methyl 2-nonynoate |
| oct-2-enoic acid ethyl ester | ethyl 2-octenoate (trans) |
| oct-2-enoic acid methyl ester | methyl 2-octenoate, (trans) |
| non-2-enoic acid methyl ester | Neofolione™ |
| hex-2-enoic acid ethyl ester | ethyl 2-hexenoate |
| hex-2-enoic acid methyl ester | methyl 2-hexenoate, methyl 3- |
| | propylacrylate |
| non-2-ynoic acid ethyl ester | ethyl 2-nonynoate |
| non-2-enoic acid ethyl ester | ethyl 2-nonenoate |
| hept-2-enoic acid ethyl ester | ethyl 2-heptenoate |
| hept-2-enoic acid methyl ester | methyl 2-heptenoate |

### Example 4a

### Validation of OMC Flavour Compositions in the Incubated Saliva (Example 3) and F. Nucleatum Test

### Example 2

OMC flavour compositions are created by mixing the ingredients as indicated in the table below. All amounts are given in % concentration (w/w). Flavour compositions containing OMC actives are designated A-F. As a comparative example, a wintergreen flavour containing no OMC actives is used (Flavour G).

| | A | B | C | D | E | F | G |
|---|---|---|---|---|---|---|---|
| Benzaldehyde | 50 | | | | | | |
| Citronellol | 100 | | | | | | |
| Eugenol | 200 | | | | | | |
| Anisaldehyde | 50 | | 10 | | | | |
| Methyl-2 butyl-isopentanoate | 80 | | | 100 | | | |
| Ethyl-2-hexenoate | 200 | 130 | 80 | 200 | 130 | 130 | |
| Ethyl-2-octenoate | 200 | 130 | 80 | 50 | 130 | 130 | |
| Methyl octinoate | 10 | 100 | 200 | 10 | 10 | 10 | |
| (Folione ™) | | | | | | | |
| Ocimene | 40 | 200 | 300 | | | | |
| Isoeugenol | 20 | | | 20 | | | |
| Dodecalactone Gamma | 50 | | | 50 | 50 | 50 | |
| Octyl acetate | | | | 400 | 580 | 280 | |
| Beta-ionone | | 80 | 80 | | 100 | 100 | |
| peppermint oil | | 360 | 250 | | | 100 | |
| Methyl salicylate | | | | | | | 700 |
| furonol | | | | | | | 20 |
| anisyl formate | | | | | | | 40 |
| Eucalyptol | | | | | | | 120 |
| wintergreen oil | | | | | | | 120 |
| Beta-cyclo Citral | | | | 100 | | | |
| cyclamen aldehyde | | | | 70 | | | |
| methyl-2-hexenoate | | | | | | 100 | |
| methyl-3-hexenoate | | | | | | 100 | |
| Anethol | | | | | | | |
| Anise oil | | | | | | | |
| Total amount OMC actives | 410 | 360 | 360 | 260 | 270 | 370 | 0 |
| according to invention | | | | | | | |
| Total amount | 1000 | 1000 | 1000 | 1000 | 1000 | 1000 | 1000 |

The OMC flavour compositions A-F and comparative example G are tested as described in examples 2 and 3 above, the results are shown in the table below. Flavours A-F are shown to provide good OMC activity even at a low concentration of 0.01% of the flavour composition yielding an end concentration of 0.0027% to 0.0041% of OMC actives. The concentration in a product needs to be 20-50 fold higher than the minimal amount for significant activity in this test to account for the dilution by salivary flow. Thus products containing 0.2-1% of a blend giving good activity at 0.01%-0.02% end concentration give sustaining efficacy when used by a consumer. Compare results in the table below. The OMC activity at a concentration of 0.02% of the OMC flavour composition containing 0.052% to 0.082% OMC actives is excellent, compare results in the table below. Additional actives present (citronellol, anisaldehyde, cyclamen aldehyde, and others classical ingredients used against oral malodour, for example peppermint oil) provide an even stronger activity.

| | | % reduction of MeSH | % reduction of MeSH |
|---|---|---|---|
| | | levels in incubated saliva | levels in incubated saliva |
| | % reduction of MeSH levels in | test, | test, |
| | cultures of F. nucleatum, | OMC flavour composition | OMC flavour composition |
| | OMC flavour composition 0.02% | 0.02% | 0.01% |
| A | 89 | >90 | 51.8 |
| B | 92 | >90 | 56 |
| C | 99 | >90 | 60 |
| D | 86 | >90 | 66 |
| E | 83 | >90 | 40.8 |
| F | 88 | >90 | 63 |
| G | <10 | 3 | 0 |
| Comparative | | | |
| wintergreen | | | |
| flavour | | | |

### Example 4b

### Mint Flavour Compositions Employing OMC Flavour Compositions A-F of Example 4a

Each OMC flavour composition A-F is combined with a mint oil (spearmint and peppermint essential oil) in a ratio of 1:1, 1:2, and 1:3 (OMC flavour composition:mint oil) as listed below.

| | A | B | C | D | E | F |
|---|---|---|---|---|---|---|
| Total amount OMC actives: | | | | | | |
| in the OMC flavour | 41% | 36% | 36% | 26% | 27% | 37% |
| compositions | | | | | | |
| 1:1 Mint essential oil | 20.5% | 18% | 18% | 13% | 13.5% | 18.5% |
| 1:2 Mint essential oil | 13.7% | 12% | 12% | — | — | 12.3% |

The OMC mint flavour compositions are effective against oral malodour and have a pleasant taste.

### Example 5

A green tea extract containing 40% epigallocatechin gallate (w/w) is tested in combination with Flavour composition A and E according example 4 in the incubated saliva test as described above. The table below lists the results obtained: At a concentration of the green tea which is insufficient for significant reduction of MeSH level (0.005%), there is a significant improvement of the activity of the flavour composition.

| | % inhibition |
|---|---|
| Treatment (% w/w in water) | MeSH formation |
| Green Tea, 0.005% | 6 |
| Flavour composition A, 0.01% | 51.8 |
| Flavour composition A, 0.01% + | 70.9 |
| Green Tea, 0.005% | |
| Flavour composition E, 0.01% | 40.8 |
| Flavour composition E, 0.01% + | 77.4 |
| Green Tea, 0.005% | |

### Example 6

### OMC Oral Care Products Comprising OMC Actives

### Example 6a

### Toothpaste, Opaque

| Ingredients | Weight % |
|---|---|
| Glycerol 98% | 3.00 |
| Thickener (Cellulose Gum CMC Blanose 7MFD, | 0.25 |
| Aqualon Company, Hercules, FR) | |
| Sorbitol 70% | 50.00 |
| Sodium Monofluorophosphate (Phoskadent Na | 0.75 |
| 211, BK Giulini Chemie, DE) | |
| Preservatives | 0.20 |
| Sodium Saccharin | 0.10 |
| Silica (Syloblanc 81) (GRACE, Germany) | 6.00 |
| Silica (Syloblanc 82) (GRACE, Germany) | 10.00 |
| Thixotropic Agent (Aerosil 200, Degussa, DE) | 2.00 |
| Titanium Dioxide (Fluka, CH) | 0.60 |
| Sodium Laurylsulfate (Fluka, CH) | 1.50 |
| Mint oil arvensis | 1% |
| OMC Flavour A according to example 4 | 0.6% |
| Purified Water | Ad 100.00 |

The concentration of OMC actives as defined herein above in the oral care product is: 0.39%. The product shows a good oral malodour counteracting effect in use.

### Example 6b

### Toothgel, Clear

| Ingredients | Weight % |
|---|---|
| Glycerol 98% | 1.60 |
| Thickener (Cellulose Gum CMC Blanose 7MFD, Aqualon | 0.30 |
| Company, Hercules, FR) | |
| Sorbitol 70% | 70.75 |
| Sodium Monofluorophosphate (Phoskadent Na 211, BK | 0.75 |
| Giulini Chemie, DE) | |
| Preservatives | 0.20 |
| Sodium Saccharin | 0.10 |
| Silica (Syloblanc 81) (GRACE, DE) | 6.00 |
| Silica (Syloblanc 82) (GRACE, DE) | 9.00 |
| Thixotropic Agent Aerosil 200, Degussa, DE | 2.00 |
| Sodium Laurylsulfate (Fluka, CH) | 1.50 |
| Mint oil arvensis | 1.00 |
| OMC Flavour E according to example 4 | 0.40 |
| Purified water | Ad 100.00 |

The concentration of OMC actives as defined herein above in the oral care product is: 0.36%. The product shows a good oral malodour counteracting effect in use.

### Example 6c

### Mouthwash

| Ingredients | Weight % |
|---|---|
| Glycerol 87% | 4.00 |
| Sorbitol 70% sol. | 8.00 |
| Sodium Saccharin | 0.01 |
| Colour 1% solution | 0.04 |
| Solubilizer Cremophor RH 410 (BASF Ltd, 67963 | 0.13 |
| Ludwigshafen, Germany) | |
| Alcohol | 7.00 |
| Mint oil | 0.16 |
| OMC Flavour A according to example 4 | 0.16 |
| Deionised Water | Ad 100.00 |

The concentration of OMC actives as defined herein above in the oral care product is: 0.1%. The product shows a good oral malodour counteracting effect in use.

### Example 6d

### Mouthspray

| Ingredients | % w/w |
|---|---|
| Solublizer Cremophor RH 410 (BASF Ltd, 67963 | 4.00 |
| Ludwigshafen, Germany) | |
| Alcohol | 30.00 |
| Glycerol 87% | 39.00 |
| Sodium Saccharin | 0.40 |
| OMC Flavour A according to example 4 | 1.00 |
| Deionised Water | Ad 100.00 |

The concentration of OMC actives as defined herein above in the oral care product is 0.65%. The product shows a good oral malodour counteracting effect in use.

### Example 6e

### Sugar Stick Chewing Gum

| Ingredients | % w/w |
|---|---|
| Gum base Valencia-T (Cafosa Gum SA., 08029 | 21.0 |
| Barcelona, Spain) | |
| Glucose syrup DE 38, 43° Bé | 18.5 |
| Icing sugar | 57.0 |
| Glycerol | 0.5 |
| Mint oil | 2.0 |
| OMC Flavour D according example 4 | 1.0 |
| Total amount | 100.0 |

The concentration of OMC actives as defined herein above in the oral care product is: 0.78%. The product shows a good oral malodour counteracting effect in use.

### Example 6f

### Sugarless Chewing Gum

| Ingredients: | % w/w |
|---|---|
| Gum base Valencia-T (Cafosa Gum SA., 08029 | 32.0 |
| Barcelona, Spain) | |
| Sorbitol powder | 47.5 |
| Lycasin concentrated | 8.0 |
| Glycerol | 1.25 |
| Mannitol powder | 4.0 |
| Xylitol milled | 4.0 |
| Aspartame | 0.2 |
| Acesulfame K | 0.05 |
| Mint oil | 2.0 |
| OMC Flavour B according to example 4 | 1.0 |
| Total amount | 100.0 |

### Example 6g

### Sugar Stick Chewing Gum

| Ingredients | % w/w |
|---|---|
| Gum base Valencia-T (Cafosa Gum SA., 08029 | 21.0 |
| Barcelona, Spain) | |
| Glucose syrup DE 38, 43° Bé | 18.5 |
| Icing sugar | 56.5 |
| Glycerol | 0.5 |
| Green tea extract synthite 1023, 40% EGCG* | 0.5 |
| Mint oil | 2.0 |
| OMC Flavour D according example 4 | 1.0 |
| Total amount | 100.0 |

*epigallocatechin gallate

The concentration of OMC actives as defined herein above in the oral care product is: 0.78%. In combination with green tea extract containing 40% EGCG, the product shows a superior oral malodour

### Example 6h

### Sugarless Chewing Gum

| Ingredients: | % w/w |
|---|---|
| Gum base Valencia-T (Cafosa Gum SA., 08029 | 32.0 |
| Barcelona, Spain) | |
| Sorbitol powder | 47.0 |
| Lycasin concentrated | 8.0 |
| Glycerol | 1.25 |
| Mannitol powder | 4.0 |
| Xylitol milled | 4.0 |
| Aspartame | 0.2 |
| Acesulfame K | 0.05 |
| Mint oil | 2.0 |
| Green tea extract synthite 1023, 40% EGCG* | 0.5 |
| OMC Flavour B according to example 4 | 1.0 |
| Total amount | 100.0 |

*epigallocatechingallate

1. A composition comprising
(a) 2 or more oral malodour counteracting actives,
wherein of said 2 oral malodour counteracting actives each has a concentration of 1% (w/w) or more based on total flavour ingredients, and
wherein the total concentration of the 2 or more oral malodour counteracting actives is from 10% (w/w) or more based on the total concentration of flavour ingredients, and
wherein each individual OMC active has a maximum concentration of up to 70% based on the total concentration of OMC actives, and
(b) optional ingredients selected from additives, excipients, solvents, and flavour ingredients;
wherein the 2 oral malodour counteracting actives are compounds according to formula I 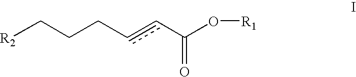
wherein
R₁ is a residue selected from the group consisting of CH₃ or CH₂CH₃;
R₂ is a residue selected from the group consisting of H, CH₃, CH₂CH₃, CH₂ CH₂CH₃;
and the double bond with dashed line as shown is a double bond or a triple bond.

2. A composition according to claim 1 wherein at least 2 of the oral malodour counteracting actives are selected from the group consisting of Oct-2-ynoic acid methyl ester, Non-2-ynoic acid methyl ester, Oct-2-enoic acid ethyl ester, Oct-2-enoic acid methyl ester, Non-2-enoic acid methyl ester, Hex-2-enoic acid ethyl ester, Hex-2-enoic acid methyl ester, Non-2-ynoic acid ethyl ester, Non-2-enoic acid ethyl ester, Hept-2-enoic acid ethyl ester, and Hept-2-enoic acid methyl ester.

3. A composition according to claim 1 further comprising one or more actives selected from the group consisting of polyphenolic compounds, polyphenolic compounds that comprise a gallate moiety, epigallocatechin gallates, green tea, green tea extract enriched in epigallocatechin gallate, ionone, alpha ionone, beta ionone, zinc salts, antibacterial agents, triclosan, cetylpyridinium chloride, polyhexidine bisguanide, chlorhexidine, antibacterial flavour materials, thymol, carvacrol, eugenol, isoeugenol, cinnamic aldehyde, menthol, essential oils containing actives including essential oils from thyme, origanum, clove, cinnamon leave, cinnamon bark, parsley seed, parsley leaf, mint, spearmint, peppermint, Octan-1-ol, 3,7-Dimethyl-oct-6-en-1-ol, 3,7-Dimethyl-octan-1-ol, 1-Isopropyl-4-methyl-cyclohex-3-enol, 3,7-Dimethyl-octa-2,6-dien-1-ol, 2-(4-Methyl-cyclohex-3-enyl) propan-2-ol, 3,7-Dimethyl-octa-1,6-dien-3-ol, Nona-2,4-dienal, Non-2-enal, 2,6,6-Trimethyl-cyclohex-1-enecarbaldehyde, 3-(4-Isopropyl-phenyl)-2-methyl-propionaldehyde, 4-Isopropenyl-cyclohex-1-enecarbaldehyde, 5-Methyl-2-phenyl-hex-2-enal, 4-Methoxy-benzaldehyde, 2,6-Dimethyl-hept-5-enal, Dec-2-enal, Phenyl-acetaldehyde, 2-Phenyl-propionaldehyde, 3,7,11-Trimethyl-dodeca-1,3,6,10-tetraene, 3,7-Dimethyl-octa-1,3,6-triene, 1-Isopropyl-4-methyl-cyclohexa-1,3-diene, 1-Methyl-4-(5-methyl-1-methylene-hex-4-enyl)-cyclohexene, 1-isopropyl-4-methylbenzene, Dec-3-en-2-one, 3-Methyl-2-pentyl-cyclopent-2-enone, 6-Methyl-hepta-3,5-dien-2-one, Acetic acid octyl ester, Acetic acid oct-2-enyl ester, 2-Methyl-but-2-enoic acid hex-3-enyl ester, Acetic acid nonyl ester, Acetic acid heptyl ester, Butyric acid 3-phenyl-allyl ester, Acetic acid 1,7,7-trimethyl-bicyclo[2.2.1]hept-2-yl ester, Acetic acid 4-allyl-2-methoxy-phenyl ester, Acetic acid 1-methyl-1-(4-methyl-cyclohex-3-enyl)-ethyl ester, Acetic acid 2-isopropenyl-5-methyl-cyclohexyl ester, 5-Octyl-dihydro-furan-2-one, 1,1-Dimethoxy-3,7-dimethyl-octa-2,6-diene, 1-Allyl-4-methoxy-benzene, 6-Hexyl-tetrahydro-pyran-2-one, 3-Butyl-3H-isobenzofuran-1-one, 2-Pentyl-furan, (2E, 5E/Z)-5,6,7-trimethylocta-2,5-dien-4-one, 4-methyl-dec-3-en-5-ol, 1-cyclopropylmethyl-4-methoxy-benzene, origanum essential oil, galbanum essential oil, litsea cubeba essential oil, tagete essential oil, jasmin absolute, lavande essential oil, lavandin essential oil, rosemary essential oil, and vetiver essential oil.

4. Composition according to claim 1, selected from the group consisting of a flavour composition, and an oral care product.

5. Oral care product according to claim 4 wherein the oral malodour counteracting actives as defined in claim 1 are in a concentration from 0.05% to 1% (w/w) based on total weight of the oral malodour counteractant oral care product.

6. Oral care product according to claim 5 selected from the group consisting of toothpaste, mouthrinse, mouthwash, chewing gum, candies, pastilles, edible films, and oral sprays.

7. Oral care product according to claim 6 wherein the oral care product is a toothpaste and wherein the oral malodour counteracting actives as defined in claim 1 are in a concentration from 0.1% to 0.625 (w/w) based on total weight of the oral malodour counteractant oral care product.

8. Oral care product according to claim 6 wherein the oral care product is a mouthrinse or mouthwash and wherein the oral malodour counteracting actives as defined in claim 1 are in a concentration from 0.05% to 0.25% (w/w) based on total weight of the oral malodour counteractant oral care product.

9. Oral care product according to claim 6 wherein the oral care product is a chewing gum and wherein the oral malodour counteracting actives as defined in claim 1 are in a concentration from 0.1% to 0.75% (w/w) based on total weight of the oral malodour counteractant oral care product.

10. A method of forming an oral malodour counteracting composition wherein 2 or more oral malodour counteracting actives as defined in claim 1 are admixed to an oral care product formulation to form an oral malodour counteracting oral care product in a concentration from 0.05% to 1% (w/w) based on total weight of the oral malodour counteractant oral care product.

11. A method of counteracting oral malodour by orally applying an oral malodour counteractant oral care product according to claim 5.

12. A method of counteracting oral malodour by orally applying an oral malodour counteractant oral care product according to claim 6.

13. A method of counteracting oral malodour by orally applying an oral malodour counteractant oral care product according to claim 7.

14. A method of counteracting oral malodour by orally applying an oral malodour counteractant oral care product according to claim 8.

15. A method of counteracting oral malodour by orally applying an oral malodour counteractant oral care product according to claim 9.

16. A method of counteracting oral malodour by orally applying an oral malodour counteractant oral care product according to claim 10.
